(19) United States
(12) Patent Application Publication
(10) Pub. No.: US 2024/0384178 A1
(43) Pub. Date: Nov. 21, 2024

(54) INTEGRATED PROCESS FOR PRODUCING LIGHT OLEFINS

(71) Applicant: UOP LLC, Des Plaines, IL (US)

(72) Inventors: Andrew B. Chin, Wilmette, IL (US); Ernest J. Boehm, Hanover Park, IL (US); Ian G. Horn, Streamwood, IL (US)

(21) Appl. No.: 18/650,018

(22) Filed: Apr. 29, 2024

Related U.S. Application Data
(60) Provisional application No. 63/466,710, filed on May 15, 2023.

(51) Int. Cl. C10G 3/00; C07C 29/15; C07C 29/80; C07C 29/92

(52) U.S. Cl. CPC C10G 3/42; C07C 29/15; C07C 29/80; C07C 29/92

(57) ABSTRACT

An integrated process for producing light olefins is disclosed. The integrated process comprises passing a syngas stream and a hydrogen stream to a methanol synthesis section to provide a reactor effluent comprising methanol. The reactor effluent is separated into a vapor stream and a liquid stream comprising methanol. The liquid stream comprising methanol is passed to a methanol purification section comprising at least two distillation columns, a first distillation column and a second distillation column to provide a methanol product stream. At least a portion of the methanol product stream is passed to an oxygenate conversion unit to provide an effluent comprising olefins. The reboiling heat to said the first distillation column and the second distillation column is provided from the separation section of the oxygenate conversion unit.

### FIELD

The field is related to an integrated process for producing light olefins. The field may particularly relate to integrating a methanol synthesis process with an oxygenate conversion process.

### BACKGROUND

Olefins have been traditionally produced from petroleum feedstock by catalytic or steam cracking processes. These cracking processes, especially steam cracking, produce light olefins such as ethylene and propylene from a variety of hydrocarbon feedstock. Ethylene and propylene are important commodity petrochemicals useful in a variety of processes for making plastics and other chemical compounds.

The petrochemical industry has known for some time that oxygenates, especially alcohols, are convertible into light olefins. For example, methanol, the preferred alcohol for light olefin production, may be converted to primarily ethylene and propylene in the presence of a molecular sieve catalyst. Molecular sieves such as microporous crystalline zeolite and non-zeolitic catalysts, particularly silicoaluminophosphates (SAPO), are known to promote the conversion of oxygenates such as methanol to light olefins. This process is referred to as a methanol-to-olefin (MTO) reaction process, which occurs in an MTO reaction system. The highly efficient Methanol to Olefin (MTO) process may convert oxygenates to light olefins which had been typically considered for plastics production. Light olefins produced from the MTO process is concentrated in ethylene and propylene but includes C4-C8 olefins.

Methanol is typically synthesized from the catalytic reaction of syngas in a methanol reactor in the presence of catalyst. Syngas is defined as a gas comprising primarily carbon monoxide (CO), hydrogen (H₂) and preferably carbon dioxide (CO₂). Other components may also be present. Syngas production processes are well known, and include conventional steam reforming, autothermal reforming, or a combination thereof.

A typical methanol synthesis system includes a light ends separation system for separating byproducts of the methanol synthesis process. Each of these separation systems may include one or more capital intensive separation units which have heating requirements and/or operations and, e.g., distillation columns, pumps and heat exchangers. Currently, the waste heat from the stripper and separator columns in the MTO process are rejected into the atmosphere through air cooling. Thus, the need exists for reducing the number equipment, the overall heat/steam utilities for the complex, and reducing overall emissions and operating costs.

### BRIEF SUMMARY

We have formulated an integrated process for producing light olefins by integrating the heat generated in a MTO process to the upstream methanol synthesis process. The integrated process can reduce the overall steam utilities for the complex, reducing overall emissions and operating costs.

### BRIEF DESCRIPTION OF THE DRAWINGS

FIG. 1 is a schematic drawing of a methanol synthesis process in accordance with an exemplary embodiment of an integrated process for producing light olefins of the present disclosure.

FIG. 2 is a schematic drawing of a methanol synthesis process in accordance with another exemplary embodiment of the integrated process for producing light olefins of the present disclosure.

FIG. 3 is a schematic drawing of a methanol synthesis process in accordance with yet another exemplary embodiment of the integrated process for producing light olefins of the present disclosure.

FIG. 4 is a schematic drawing of a MTO process in accordance with an exemplary embodiment of an integrated process for producing light olefins of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

As used herein, the term “predominant” or “predominate” means greater than 50%, suitably greater than 75% and preferably greater than 90%.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column. Stripping columns may omit a reboiler at a bottom of the column and instead provide heating requirements and separation impetus from a fluidized inert media such as steam. Stripping columns typically feed a top tray and take main product from the bottom.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure. As used herein, the term “boiling point temperature” means atmospheric equivalent boiling point (AEBP) as calculated from the observed boiling temperature and the distillation pressure, as calculated using the equations furnished in ASTM D1160 appendix A7 entitled “Practice for Converting Observed Vapor Temperatures to Atmospheric Equivalent Temperatures”.

As used herein, the term “True Boiling Point” (TBP) means a test method for determining the boiling point of a material which corresponds to ASTM D-2892 for the production of a liquefied gas, distillate fractions, and residuum of standardized quality on which analytical data can be obtained, and the determination of yields of the above fractions by both mass and volume from which a graph of temperature versus mass % distilled is produced using fifteen theoretical plates in a column with a 5:1 reflux ratio.

As used herein, the term “T5”, “T10”, “T90” or “T95” means the temperature at which 5 mass percent, 10 mass percent, 90 mass percent or 95 mass percent, as the case may be, respectively, of the sample boils using ASTM D-86 or TBP.

As used herein, the term “initial boiling point” (IBP) means the temperature at which the sample begins to boil using ASTM D-7169, ASTM D-86 or TBP, as the case may be.

As used herein, the term “end point” (EP) means the temperature at which the sample has all boiled off using ASTM D-7169, ASTM D-86 or TBP, as the case may be.

As used herein, the term “diesel” means hydrocarbons boiling in the range of an IBP between about 125° C. (257° F.) and about 175° C. (347° F.) or a T5 between about 150° C. (302° F.) and about 200°° C. (392° F.) and the “diesel cut point” comprising a T95 between about 343° C. (650° F.) and about 399° C. (750° F.) using the TBP distillation method or a T90 between 280° C. (536° F.) and about 340°° C. (644° F.) using ASTM D-86. The term “green diesel” means diesel comprising hydrocarbons not sourced from fossil fuels.

As used herein, the term “jet fuel” means hydrocarbons boiling in the range of a T10 between about 190° C. (374° F.) and about 215° C. (419° F.) and an end point of between about 290° C. (554° F.) and about 310° C. (590° F.). The term “green jet fuel” or “renewable jet fuel” means jet fuel comprising hydrocarbons not sourced from fossil fuels.

As used herein, the term “a component-rich stream” means that the rich stream coming out of a vessel has a greater concentration of the component than the feed to the vessel and preferably than all other streams withdrawn from the vessel.

As used herein, the term “a component-lean stream” means that the lean stream coming out of a vessel has a smaller concentration of the component than the feed to the vessel and preferably than all other streams withdrawn from the vessel.

### DETAILED DESCRIPTION

An integrated process and apparatus for producing light olefins is disclosed. The integrated process and apparatus disclosed herein involves integrating the heat generated in a MTO process to the upstream methanol synthesis process. A substantially heated stream of the MTO process may be utilized in the methanol synthesis process to provide heat duty of various units and/or columns. The one or more streams from the MTO process may be pumped to the Methanol Synthesis and/or Purification units upstream and cross exchange with the process as feed preheat, reboiler heat, etc. The stream after heat exchange will circulate back to their respective columns and/or units.

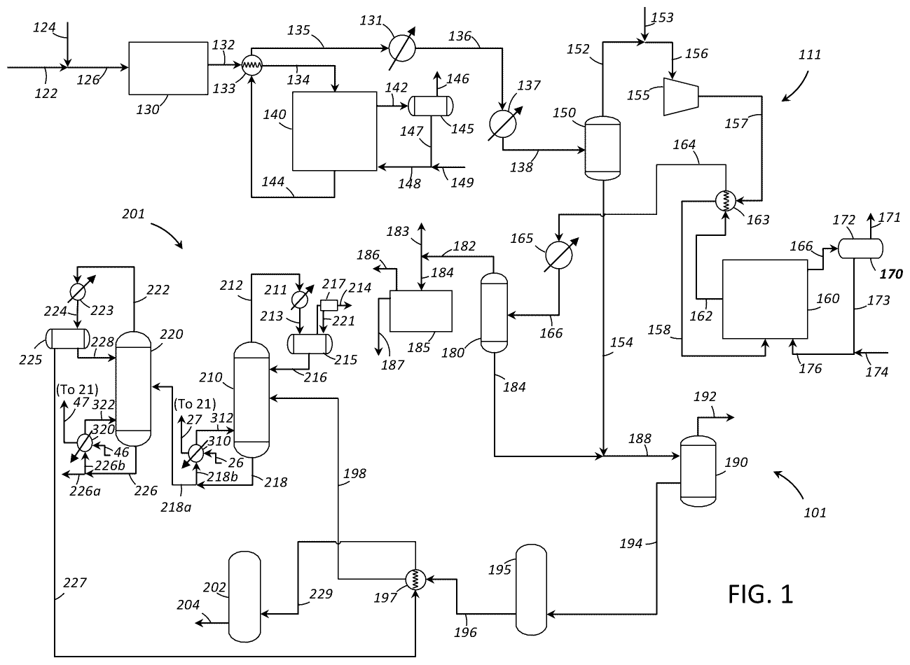

Turning to FIG. 1 of an integrated process and apparatus 101 for producing light olefins comprises a methanol synthesis section 111 and a methanol purification section 201. As shown in FIG. 1, a syngas stream in line 122 and a hydrogen gas stream in line 124 are passed to the methanol synthesis section 111. Syngas is defined as a gas comprising primarily carbon monoxide (CO), carbon dioxide (CO2) and hydrogen (H2). Optionally, syngas may also include methane (CH4), and small amounts of ethane and propane. Conventional processes for converting carbon components to syngas include steam reforming, partial oxidation, autothermal reforming, and combinations of these processes. In accordance with an embodiment of the present disclosure, the syngas stream in line 122 may be taken from any suitable sources. In accordance with another embodiment of the present disclosure, the hydrogen gas stream in line 124 may be taken from any suitable sources. In an exemplary embodiment, the hydrogen gas stream in line 124 is taken from a pressure swing adsorption (PSA) unit. In another exemplary embodiment, the hydrogen gas stream in line 124 may be taken from a water electrolysis unit.

In accordance with an exemplary embodiment, of the present disclosure, the methanol synthesis section 111 comprises a first methanol converter 140 and a second methanol converter 160. The syngas stream in line 122 and the hydrogen gas stream in line 124 are passed to the first methanol converter 140 of the methanol synthesis section 111. In an embodiment, the syngas stream in line 122 and the hydrogen gas stream in line 124 may be combined to provide a combined feed stream 126 which is passed to the first methanol converter 140. However, the syngas stream in line 122 and the hydrogen gas stream in line 124 may be passed separately to the first methanol converter 140. The combined feed stream 126 may be passed to a syngas pressure booster compressor 130 to compress the syngas to a particular pressure to provide a compressed syngas stream in line 132 before passing to the first methanol converter 140. In an exemplary embodiment, the syngas may be compressed to a pressure from about 6890 kPa (1000 psia) to about 8970 kPa (1300 psia) in the syngas pressure booster compressor 130. In an embodiment, the syngas pressure booster compressor 130 comprises a multistage compressor. In an embodiment, the syngas pressure booster compressor 130 comprises a two-stage compressor. The syngas stream may be heated before passing to the first methanol converter 140. The compressed syngas stream in line 132 may be heat exchanged in a heat exchanger 133 to provide a heated syngas stream in line 134. The heated syngas stream in line 134 is passed to the first methanol converter 140.

In the first methanol converter 140 of the methanol synthesis section 111, the syngas is converted to a methanol composition. The methanol synthesis process is accomplished in the presence of a methanol synthesis catalyst. In an exemplary embodiment, the syngas stream in line 122 to the methanol synthesis section 111 has a molar ratio of carbon dioxide to carbon monoxide of between 1:2 and 1:4 and a molar ratio of hydrogen to carbon oxides (carbon monoxide and carbon dioxide) in the range of from about 3:2 to about 3:1.

A suitable methanol synthesis catalyst may be a copper on a zinc oxide and alumina support. Synthesis conditions of the first methanol converter 140 of the methanol synthesis section 111 may include a temperature of about 200 to about 300° C. and a pressure of about 3.5 to about 10 MPa. Reaction equilibrium typically requires methanol separation and recycle of unreacted reagents to the synthesis reaction.

In accordance with an exemplary embodiment, the first methanol converter 140 is operating at a temperature of about 204° C. (400° F.) to about 290° C. (550° F.). In accordance with another exemplary embodiment, the first methanol converter 140 is operated at a pressure from about 6890 kPa (1000 psia) to about 8970 kPa (1300 psia).

The methanol synthesis reaction is highly exothermic. A boiler feed water (BFW) in line 148 is passed to the first methanol converter 140 for heat exchange with the process stream(s) and to generate a steam stream which is withdrawn in line 142 from the first methanol converter 140. In an aspect, the boiler feed water (BFW) in line 148 is passed to a heat exchanger of the first methanol converter 140 to absorb heat and produce steam in line 142. The generation of steam absorbs the exotherm in the methanol synthesis reaction. The steam stream in line 142 is passed to an overhead separator 145 to separate steam in line 146 from a water stream in line 147. The water stream in line 147 is supplemented with a recycled BFW in line 149 to provide the BFW in line 148 for the first methanol converter 140.

In the first methanol converter 140, the syngas is converted to a methanol composition in a first reactor effluent stream comprising methanol in line 144. The methanol stream in the first reactor effluent stream in line 144 may include methanol, dimethyl ether, ethanol or combinations thereof. The first reactor effluent stream in line 144 is heat exchanged in the heat exchanger 133 with the compressed syngas stream in line 132 to provide a first cooled first reactor effluent stream in line 135. The first cooled first reactor effluent stream in line 135 may be cooled in a cooler 131 to provide a second cooled first reactor effluent stream in line 136. The second cooled first reactor effluent stream in line 136 may be further cooled in a cooler 137 to provide a third cooled first reactor effluent stream in line 138. The third cooled first reactor effluent stream in line 138 is separated in a first gas-liquid separator 150 to provide a first vapor stream in line 152 and a first liquid stream in line 154. The first vapor stream in line 152 and the first liquid stream in line 154 may be further processed to recover methanol.

The first vapor stream in line 152 comprises one or more carbon oxide that has not yet converted to methanol. The first vapor stream in line 152 may be compressed in a first compressor 155. In an embodiment, the first vapor stream in line 152 may be combined with a make-up hydrogen stream in line 153 to provide a combined first vapor stream in line 156. The combined first vapor stream in line 156 is compressed in the first compressor 155 to provide a compressed first vapor in line 157 at a pressure from about 6890 kPa (1000 psia) to about 8970 kPa (1300 psia). In an embodiment, the make-up hydrogen stream in line 153 may be taken from any suitable sources. In accordance with the present disclosure, the make-up hydrogen stream in line 153 may be taken from one or more of the units of the process 101.

The compressed first vapor in line 157 is heat exchanged with a reactor effluent in the heat exchanger 163 to provide a heat exchanged first vapor stream in line 158 which is passed to the second methanol converter 160. In the second methanol converter 160 of the methanol synthesis section 111, the unconverted carbon dioxide in the syngas is converted to a methanol composition. The methanol synthesis process is accomplished in the presence of a methanol synthesis catalyst. A suitable methanol synthesis catalyst may be a copper on a zinc oxide and alumina support. Synthesis conditions of the second methanol converter 140 of the methanol synthesis section 111 may include a temperature of about 200 to about 300° C. and a pressure of about 3.5 to about 10 MPa. Reaction equilibrium typically requires methanol separation and recycle of unreacted reagents to the synthesis reaction.

A boiler feed water (BFW) in line 176 is passed to the second methanol converter 160 for heat exchange with the process stream(s) to absorb the exotherm and to generate a steam stream which is withdrawn in line 166 from the second methanol converter 160. In an aspect, the boiler feed water (BFW) in line 176 is passed to a heat exchanger of the second methanol converter 160 to absorb heat and produce steam in line 166. The steam stream in line 166 is passed to an overhead separator 172 to separate steam in line 171 from a water stream in line 173. The water stream in line 173 is supplemented with a recycled BFW in line 174 to provide the BFW in line 176 for the second methanol converter 160.

In the second methanol converter 160, the first reactor effluent is converted to a methanol composition to provide a second reactor effluent stream comprising methanol in line 162. The methanol stream in the second reactor effluent stream in line 162 may include methanol, dimethyl ether, ethanol or combinations thereof. The second reactor effluent stream in line 162 is withdrawn from the second methanol converter 160. The second reactor effluent stream in line 162 is heat exchanged in the heat exchanger 163 with the compressed first vapor in line 157 to provide a first cooled second reactor effluent stream in line 164. The first cooled second reactor effluent stream in line 164 may be cooled in a cooler 165 to provide a second cooled second reactor effluent stream in line 166. The second cooled second reactor effluent stream in line 166 is separated in a second gas-liquid separator 180 to provide a second vapor stream in line 182 and a second liquid stream in line 184. The second vapor stream in line 182 and the second liquid stream in line 184 may be further processed to recover methanol.

In accordance with an exemplary embodiment, the second methanol converter 160 is operating at a temperature of about 204° C. (400° F.) to about 290° C. (550° F.). In accordance with another exemplary embodiment, the second methanol converter 160 is operated at a pressure from about 6890 kPa (1000 psia) to about 8970 kPa (1300 psia).

In accordance with the present disclosure, the second vapor stream in line 182 is passed to a PSA unit 185 to separate hydrogen from the second vapor stream in line 182. In an exemplary embodiment, the second vapor stream in line 182 may be separated into a recycle stream in line 183 and a PSA feed stream in line 184. In another exemplary embodiment, the recycle stream in line 183 may be passed to the first compressor 155 as the make-up hydrogen stream. In an embodiment, the make-up hydrogen stream in line 153 to the first compressor 155 comprises the recycle stream in line 183.

The PSA feed stream in line 184 is processed in the PSA unit 185. Typically, PSA unit includes a series of multiple adsorber beds containing one or a combination of multiple adsorbents suitable for adsorbing the particular components to be adsorbed therein. These adsorbents include, but are not limited to, activated alumina, silica gel, activated carbon, zeolite molecular sieve type materials, or any combination thereof. The adsorbents are organized in any sequence as required by the adsorption process to adsorb impurities or components. In the PSA unit 185, PSA feed gas flows over the adsorbents and the more readily adsorbable impurities are adsorbed during the adsorption step while hydrogen flows through. Pressure swing enables adsorbed impurities on the adsorbent to desorb into line 186. The purified hydrogen gas leaves the adsorber bed in the PSA hydrogen stream 187 that is lean in impurities.

In the PSA unit 185, hydrogen present in the PSA feed stream in line 184 is separated. As shown, from the PSA unit 185, a purge stream in line 186 is separated from a hydrogen rich stream in line 187. The purge stream in line 186 may be used as fuel. In an exemplary embodiment, the hydrogen rich stream in line 187 may be passed to the syngas pressure booster compressor 130 as a portion of the hydrogen stream in line 124. In an embodiment, the hydrogen stream in line 124 to the syngas pressure booster compressor 130 comprises the hydrogen rich stream in line 187.

Turning back to the second gas-liquid separator 180, the second liquid stream in line 184 is withdrawn from the bottoms of the second gas-liquid separator 180 and passed to a third gas-liquid separator 190. The first liquid stream in line 154 may also be passed to the second gas-liquid separator 180. In an exemplary embodiment, the second liquid stream in line 184 may be combined with the first liquid stream in line 154 to provide a combined liquid stream in line 188 which is passed to the third gas-liquid separator 190. In the third gas-liquid separator 190, the first liquid stream in line 154 and the second liquid stream in line 184 are separated into a third vapor stream in line 192 and a third liquid stream in line 194. The third vapor stream in line 192 comprises light ends, and it may be passed to the fuel gas system. The third liquid stream in line 194 comprises crude methanol. Alternately, the third liquid stream in line 194 may be a crude methanol stream. The crude methanol stream may comprise at least 100 ppmw of carbon oxide and/or at least 100 ppmw C2+ oxygenates.

The crude methanol comprises methanol, light ends, and heavier alcohols. As used and described herein, the term “crude methanol” or “crude oxygenate feedstock” may comprise methanol, ethanol, water, light ends, and fusel oil. The light ends may include ethers, ketones, aldehydes, and dissolved gases such as hydrogen, methane, carbon oxide, and nitrogen. The crude methanol comprises fusel oil. The fusel oil in the crude methanol typically includes higher molecular weight alcohols and is generally burned as a fuel in the methanol plant. The crude methanol comprising the fusel oil can be passed to the oxygenate conversion unit for the additional production of light olefins. The crude methanol may be passed to the oxygenate conversion unit or the MTO unit for feed.

In accordance with an exemplary embodiment of the present disclosure, the crude methanol may have a composition comprising carbon monoxide (CO) in a concentration from about 0 to about 1 wt %, carbon dioxide (CO₂) in a concentration from about 0.05 wt % to about 2 wt %, methane (CH₄) in a concentration from about 0.001 wt % to about 2 wt %, hydrogen (H₂) in a concentration from about 0.05 wt % to about 2 wt %, oxygen (O₂) in a concentration from about 0 to about 1 wt %, water (H2O) in a concentration from about 5 wt % to about 18 wt %, nitrogen (N₂) in a concentration from about 0 to about 1 wt %, methanol (CH₃OH) in a concentration from about 75 wt % to about 90 wt %, and alcohols (other than methanol) in a concentration from about 0.05 to about 4 wt %.

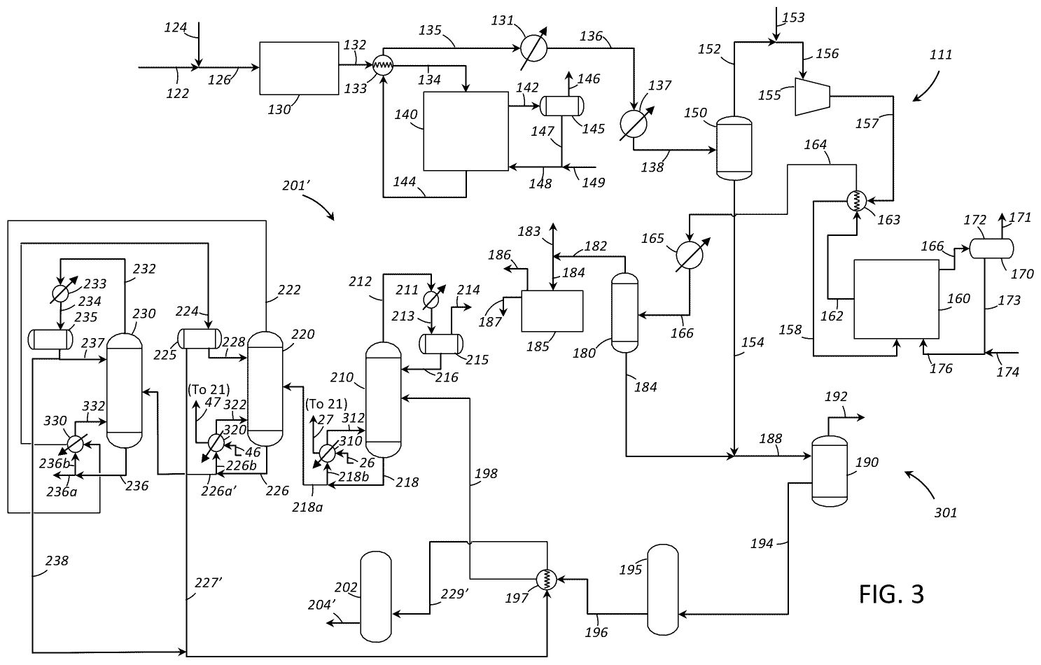

The third liquid stream in line 194 may be passed to a crude methanol hold-up tank 195 which could be a pressurized vessel or a tank. A crude methanol stream in line 196 is withdrawn from the crude methanol hold-up tank 195. In accordance with the present disclosure, the crude methanol stream in line 196 may be passed to the oxygenate conversion unit 110 as shown in FIG. 3. In accordance with an embodiment, the crude methanol stream in line 196 may be passed to a methanol purification section 201 to separate by-products and provide a methanol product stream for the oxygenate conversion unit 10.

In accordance with an exemplary embodiment, the crude methanol stream in line 196 may be passed to the methanol purification section 201 comprising at least two distillation columns, a first distillation column 210 and a second distillation column 220. The crude methanol stream in line 196 is heat exchanged with a product stream in a heat exchanger 197 to provide a heat exchanged perhaps a heated crude methanol stream in line 198. The heated crude methanol stream in line 198 is passed to the first distillation column 210. In the first distillation column 210, the light gas(es) are separated from the crude methanol in a first distillation column overhead stream in line 212. The light gases separated from the crude methanol stream include carbon monoxide, carbon dioxide, methane, hydrogen and dimethyl ether. The first distillation column overhead stream in line 212 is passed to a first overhead condenser 211 to provide a partially condensed first distillation column overhead stream in line 213, which is then passed to a first overhead receiver 215 where the light gas(es) is separated in a first overhead receiver vapor stream in line 214. The first overhead receiver vapor stream in line 214 may be passed to a fuel gas section or used as a fuel. From the first overhead receiver 215, an overhead receiver liquid stream is withdrawn in line 216 and passed to a top of the first distillation column 210.

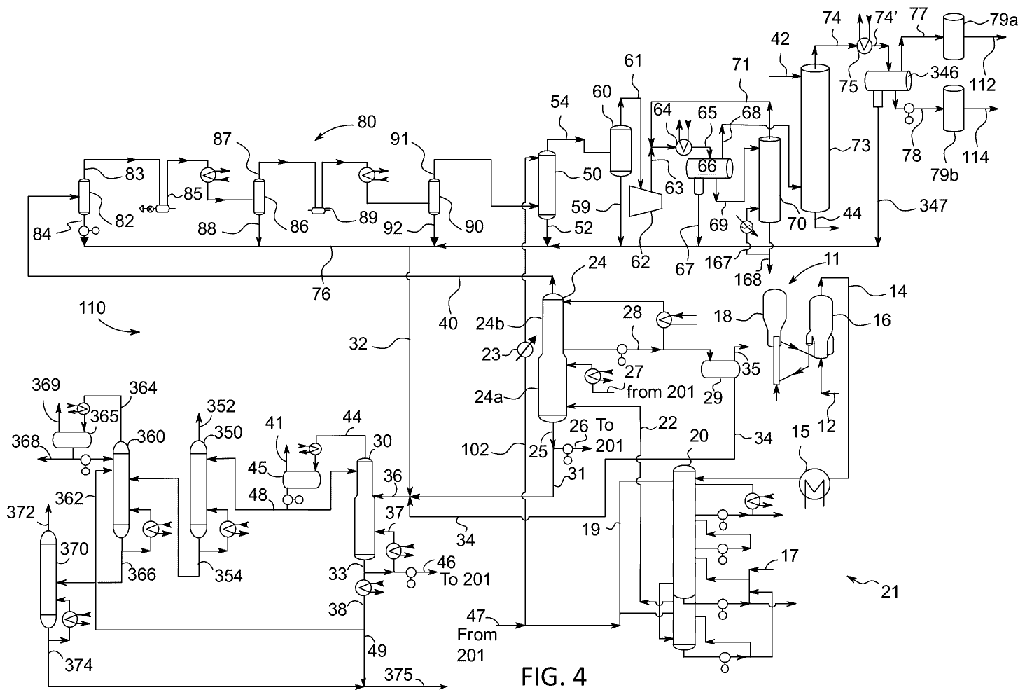

A first distillation column bottoms stream comprising methanol in line 218 is withdrawn for further separation. The first distillation column bottoms stream in line 218 is separated into a first reboiling stream in line 218b and a first distillation column effluent stream in line 218a. The first reboiling stream in line 218b is reboiled in a reboiler 310 before passing to the first distillation column bottom section. Typically, the reboilers account for significant heat duty consumption and associated operating costs. Applicants have found that the first reboiling stream in line 218b can be reboiled in the reboiler 310 by heat exchange with a suitable stream from the downstream oxygenate conversion unit providing the requisite heating in the reboiler 310. Applicants found that a water rich stream from the separation section 21 of the downstream oxygenate conversion unit 110 in FIG. 4 can be suitably used for reboiling the first reboiling stream in line 218b in the reboiler 310. Accordingly, the first reboiling stream in line 218b is heated/reboiled in the reboiler 310 of the first distillation column 210 with a product water stream in line 26 of the separation section 21 to provide a first reboiled stream in line 312 and a cooled product water stream in line 27. The cooled product water stream in line 27 is circulated back to separation section 21 of the downstream oxygenate conversion unit.

The first distillation column effluent stream in line 218a includes heavy oxygenates such as C2+ alcohols, ketones, aldehydes that should be removed from the crude methanol stream. Hence the first distillation column effluent stream in line 218a is further separated in a second distillation column 220. In the second distillation column 220, the first distillation column effluent stream in line 218a is separated into a second distillation column overhead stream in line 222 comprising methanol and a second distillation column bottoms stream in line 226. The second distillation column overhead stream in line 222 is passed through a second overhead condenser 223 to provide a condensed second distillation column overhead stream in line 224, which is passed to a second overhead receiver 225. In the second overhead receiver 225, the condensed second distillation column overhead stream in line 224 is separated into a second reflux stream in line 228 and a methanol product stream in line 227. The second reflux stream in line 228 is recycled to the second distillation column 220. The methanol product stream in line 227 is a first methanol product stream from the process 101.

In accordance with an exemplary embodiment, the first distillation column 210 is operated at a pressure from about 138 kPa (20 psia) to about 1379 kPa (200 psia). In accordance with another exemplary embodiment, the first distillation column is operated at a temperature of about −17° C. (0° F.) to about 177° C. (350° F.).

A second distillation column bottoms stream in line 226 is withdrawn from the column. The second distillation column bottoms stream in line 226 is separated into a second reboiling stream in line 226b and a second distillation column effluent stream in line 226a. The second reboiling stream in line 226b is reboiled in a reboiler 320 before passing it to the second distillation column bottom section. Applicants found that the second reboiling stream in line 226b can be reboiled in the reboiler 320 by heat exchange with another suitable stream from the downstream oxygenate conversion unit providing the requisite heating in the reboiler 320. Applicants found that a stripped water stream in line 46 from the separation section 21 of the downstream oxygenate conversion unit 110 in FIG. 4 can be suitably used for reboiling the second reboiling stream in line 226b in the reboiler 320. Accordingly, the second reboiling stream in line 226b is heated/reboiled in the reboiler 320 of the second distillation column 220 with a circulating stripped water stream in line 46 of the separation section 21 to provide a second reboiled stream in line 322 and a cooled circulating stripped water stream in line 47. The cooled stripped water stream in line 47 is circulated back to separation section 21 of the downstream oxygenate conversion unit. In an aspect, another stream or heat source may be passed to the reboiler 320 or a supplementary reboiler (not shown) to provide sufficient additional heat to reboil the second reboiling stream in line 226b.

In accordance with an exemplary embodiment, the second distillation column is operating at a pressure from about 35 kPa (5 psia) to about 862 kPa (125 psia). In accordance with yet an exemplary embodiment, the second distillation operates at a temperature of about 38° C. (100° F.) to about 149°° C. (300° F.).

The two distillation columns, the first distillation column 210 and the second distillation column 220 as shown in FIG. 1 may be operated at a lower pressure. Under low pressure operations, the water rich streams from the separation section 21 of the downstream oxygenate conversion unit 110 in FIG. 4 can be suitably used for reboiling the first reboiling stream in line 218b and the second reboiling stream in line 226b. The embodiment shown in FIG. 1 may apply to a low-pressure mode operation of the first distillation column 210 and the second distillation column 220.

In an aspect, the first overhead receiver vapor stream in line 214 may be passed to a vent condenser 217 to increase the recovery from the first distillation column overhead when operating in low pressure mode. Condensate from the vent condenser 217 may be returned to the first overhead receiver 215 in line 221.

Referring back to the second distillation column 220, the methanol product stream in line 227, after heat exchange with the crude methanol stream in line 196 in the heat exchanger 197 to provide a cooled methanol product stream in line 229, is passed to a methanol product hold-up tank 202. A methanol product stream in line 204 is withdrawn from the methanol product hold-up tank 202 for further processing as disclosed herein after in detail.

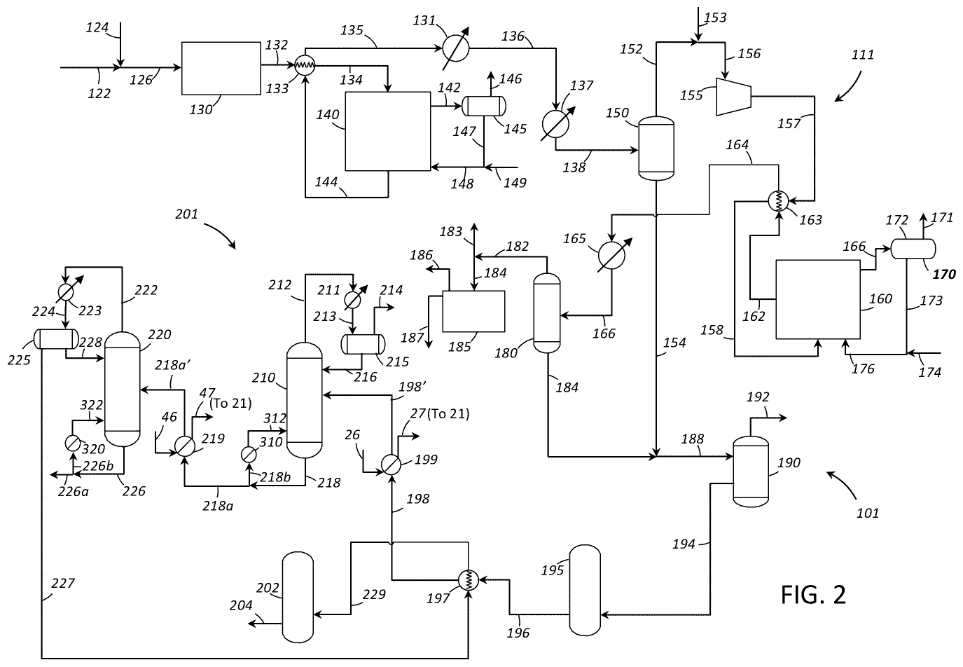

FIG. 2 shows an alternative embodiment to the embodiment of FIG. 1. Elements in FIG. 2 with the same configuration as in FIG. 1 will have the same reference numeral as in FIG. 1. Elements in FIG. 2 which have a different configuration as the corresponding element in FIG. 1 will have the same reference numeral but designated with a prime symbol (′). The configuration and operation of the embodiment of FIG. 2 is essentially the same as in FIG. 1 with the following exceptions.

The two distillation columns, the first distillation column 210 and the second distillation column 220, may be operated at a higher pressure. Under high pressure operation, the water rich streams from the separation section 21 of the downstream oxygenate conversion unit 110 in FIG. 4 can be suitably used for heating the feed to the first distillation column 210 and the second distillation column 220. The embodiment shown in FIG. 2 may apply to a high-pressure mode operation of the first distillation column 210 and the second distillation column 220.

The heated crude methanol stream in line 198 may be heat exchanged with the water rich streams from the separation section 21 of the downstream oxygenate conversion unit 110 in FIG. 4 in a heat exchanger 199. In an embodiment, the heated crude methanol stream in line 198 may be further heated in the heat exchanger 199 by heat exchange with the product water stream in line 26 of the separation section 21 to provide a further heated crude methanol stream in line 198′ and a cooled product water stream in line 27. The cooled product water stream in line 27 is circulated back to separation section 21 of the downstream oxygenate conversion unit 110 in FIG. 4. The further heated crude methanol stream in line 198′ is passed to the first distillation column 210.

The first distillation column bottoms stream in line 218 is separated into the first reboiling stream in line 218b and the first distillation column net bottoms effluent stream in line 218a. The first reboiling stream in line 218b is heated in the reboiler 310 to provide the first reboiled stream in line 312 which is recycled to the bottom of the first distillation column 210.

In an aspect, the first overhead receiver vapor stream in line 214 may be passed to a vent condenser 217 to increase the recovery from the first distillation column overhead when operating in low pressure mode. Condensate from the vent condenser 217 may be returned to the first overhead receiver 215 in line 221.

The net bottoms effluent stream in line 218a from the first distillation column 210 is passed to the second distillation column 220. In an embodiment, the first distillation column effluent stream in line 218a may be heated in a heat exchanger 219 by heat exchange with a circulating stripped water stream in line 46 of the separation section 21 to provide a heated first effluent stream in line 218a′ and a cooled circulating stripped water stream in line 47. The cooled stripped water stream in line 47 is circulated back to the separation section 21 of the downstream oxygenate conversion unit. The heated first effluent stream in line 218a′ is passed to the second distillation column 220.

The second distillation column bottoms stream in line 226 is separated into the second reboiling stream in line 226b and a second distillation column net bottoms effluent stream in line 226a. The second reboiling stream in line 226b is reboiled in the reboiler 320 to provide the second reboiled stream in line 322 which is recycled to the bottom of the second distillation column 220.

FIG. 3 shows another alternative embodiment to the embodiment of FIG. 1. Elements in FIG. 3 with the same configuration as in FIG. 1 will have the same reference numeral as in FIG. 1. Elements in FIG. 3 which have a different configuration as the corresponding element in FIG. 1 will have the same reference numeral but designated with a prime symbol (′). The configuration and operation of the embodiment of FIG. 3 is essentially the same as in FIG. 1 with the following exceptions.

In accordance with an embodiment of the present disclosure, the methanol purification section 201 also comprises a third distillation column 230 for further removing heavy oxygenates from the crude methanol stream. In accordance with an exemplary embodiment, the third distillation column 230 may operate at a pressure from about 35 kPa (5 psia) to about 345 kPa (50 psia). In accordance with another exemplary embodiment of the present disclosure, the third distillation column 230 may be an atmospheric column operating at about atmospheric pressure. In accordance with yet an exemplary embodiment, the third distillation column 230 is operated at a temperature of about 38° C. (100° F.) to about 122° C. (250° F.).

When the third column 230 is employed, the second distillation column effluent stream in line 226a′ is separated in the third distillation column to provide a third distillation column overhead stream in line 232 comprising methanol and a third distillation column bottoms stream in line 236. The third distillation column overhead stream in line 232 is condensed in a third overhead condenser 233 and a condensed third distillation column overhead stream in line 234 is passed to a third overhead receiver 235. A third distillation column reflux stream is taken in line 237 from the third overhead receiver 235. A methanol product stream is taken in line 238 from the third overhead receiver 235. The methanol product stream in line 238 from the third distillation column 230 is a second methanol product stream from the process 101. The second methanol product stream in line 238 is taken from the third overhead receiver 235 and passed to the methanol product hold-up tank 202 along with the methanol product stream in line 227′. The third distillation column bottoms stream in line 236 is depleted of methanol. The third distillation column bottoms stream in line 236 is withdrawn from the third distillation column 230 and split into a third distillation column reboiling stream in line 236b and a third distillation column bottoms effluent stream in line 236a. The third distillation column reboiling stream in line 236b is heated in a third distillation column reboiler 330 and a third reboiled stream in line 332 is returned to the third distillation column 230.

In accordance with an exemplary embodiment of the present disclosure, the second distillation column overhead stream in line 222 may be passed to the third distillation column reboiler 330 to provide heat to the third distillation column reboiling stream in line 236b. A third reboiled stream in line 332 is passed back to the third distillation column bottom and a condensed second distillation column overhead stream is taken in line 224 and passed to the second overhead receiver 225.

The second methanol product stream in line 238 from the third distillation column 230 may be combined with the methanol product stream in line 227 from the second distillation column 220 to provide a combined methanol product stream in line 229′ which is passed to the methanol product hold-up tank 202. The methanol product stream may be taken in line 204′ from the product hold-up tank 202 and processed in the oxygenate conversion section 11.

In accordance with another embodiment of the present disclosure, the integrated process and apparatus for producing light olefins comprises an oxygenate conversion unit 110 or an MTO unit 110 as shown in FIG. 4. The oxygenate conversion unit 110 may comprise an MTO unit which includes an oxygenate conversion section 11, a separation section 21, and a compression section 80. The methanol product stream in line 204 may be processed in the oxygenate conversion section 11. Alternately, the crude methanol stream in line 196 can be processed in the oxygenate conversion section 11. The oxygenate conversion unit 110 comprises a separation section 21 comprising a quench column 20, a product separator column 24, a water stripper column 30, a DME stripper column 350, an extractive distillation column 360, and a methanol stripper 370.

As shown in FIG. 4, the oxygenate conversion section 11 comprises an oxygenate conversion reactor 16 that reacts oxygenates such as methanol or dimethyl ether (DME) with fluidized catalyst. A superheated feed stream in line 12 comprising a methanol product stream in line 204 or the crude methanol stream in line 196 is fed to the oxygenate conversion reactor 16. The oxygenate conversion reactor 16 may comprise fluidized catalyst at fast fluidized conditions. The catalysts may be a silicoaluminophosphate (SAPO) catalyst. SAPO catalysts and their formulation are generally taught in U.S. Pat. Nos. 4,499,327A, 10,358,394 and 10,384,986.

A hot vaporous reactor effluent stream in line 14 is withdrawn from the oxygenate conversion reactor 16 which periodically or continuously circulates fluidized catalyst in a conventional manner to a regeneration zone 18 to maintain the selectivity and the conversion desired. Reactor 16 is maintained at effective conditions for the conversion of the oxygenate/methanol to produce light olefin products and generate oxygenated byproducts. The hot vaporous reactor effluent stream may comprise light olefins, water, and oxygenates.

The hot vaporous reactor effluent stream in line 14 may be preliminarily cooled in a reactor effluent heat exchanger 15 to recover heat before it is passed to a quench column 20. In the quench column 20, the vaporous reactor effluent is desuperheated, neutralized of organic acids and clarified of catalyst fines by direct contact with a water stream supplied in line 19 which may be taken from a cooled circulating stripped water stream in line 47. In addition, circulated water streams in the quench tower system are employed in multiple stages to enhance catalyst fines recovery. An additional section in the quench column 20 may be provided for caustic injection to remove organic acids such as acetic acid and entrained caustic from a caustic contacting section. A caustic stream in line 17 may be injected into the quench column 20. If the quench column 20 includes a caustic section, it may also include a water wash section to remove caustic in counter current flow from a quenched stream. A quenched olefin stream in line 22 is discharged from the quench column 20 and fed to the product separator column 24 in the separation section 21.

The product separator column 24 comprises two sections for separating the reactor effluent stream into a product olefin stream in an overhead line 40, an intermediate liquid stream in an intermediate line 28 and a water stream in a bottoms line 25. The water stream in the bottoms line 25 may be separated into a circulating product water stream in bottoms line 26 and a product water stream in a net bottoms line 31. The circulating product water stream in the bottoms line 26 may be a first pump-around stream of the product separator column 24. A first section 24a receives the quenched reactor effluent stream in line 22. In the first section 24a, most of the heat is removed from the quenched reactor effluent stream while partially condensing the water in the quenched reactor effluent stream to generate the product water stream in the bottoms line 25 comprising a portion of the oxygenate byproducts in the quenched reactor effluent stream in line 22. A pump-around product water stream may be cooled and pumped around to the top of the first section 24a of the product separator column 24 in the quenched reactor effluent stream in line 27. The product water stream in line 31 is fed to the water stripper column 30.

As disclosed herein above, the applicants found that the circulating product water stream in line 26 from the separation section 21 of the oxygenate conversion unit 110 can be suitably used for reboiling the first reboiling stream in line 218b in the reboiler 310 from the bottoms line 218 of the first distillation column 210 of the of the methanol purification unit 201 in FIGS. 1 and 3. Thus, the product water stream in line 26 is passed to the reboiler 310 to heat the first reboiled stream in line 312 and provide the cooled product water stream in line 27 as shown in FIGS. 1 and 3. Alternatively, the circulating product water stream in line 26 could preheat the feed to the first distillation column 220 in line 198 as shown in FIG. 2. The cooled product water stream in line 27 is circulated back to the product separator column 24 of the separation section 21.

In accordance with an embodiment of the present disclosure, the cooled product water stream in line 27 after heating the reboil stream in line 218b or other stream in the methanol purification unit 201 returns cooled in line 27 and is passed to a water stripper column 30. Preferably, the cooled product water stream in line 27 is returned to the product separator column 24 to the top of the first section 24a. In an embodiment, the cooled product water stream in line 27 is returned to the product separator column 24 upstream of the cooler in line 27. The water stripper column 30 may be in downstream communication with the product separator column 24 and in upstream communication with the compression section 80.

A vapor stream from the first section 24a of the product separator column 24 is passed to the second, or upper, section 24b of the product separator. An intermediate stream in line 28 comprising hydrocarbons, oxygenate byproducts, and water in liquid phase is withdrawn at a bottom of the upper section 24b. The intermediate stream in line 28 may be a second pump-around stream of the product separator column 24. A portion of the intermediate stream in line 28 is cooled and passed as cooled contacting fluid to the top of the second section of the product separator column 24. The remainder of the intermediate stream in line 28 is passed to a coalescer 29 to separate a hydrocarbon overhead stream in line 35 from an aqueous stream in line 34 which may be fed to the water stripper column 30. An overhead product olefin stream comprising olefins from the second section 24b of the product separator column 24 in line 40 is delivered to the compression section 80. In accordance with an exemplary embodiment, the cooled product water stream in line 27, the aqueous stream in line 34, and a water return stream comprising oxygenate byproducts from the compression section 80 in return line 32 are combined to provide a combined product water stream in line 36. The combined product water stream in line 36 is passed to the water stripper column 30. Also, the cooled product water stream in line 27, the aqueous stream in line 34, and the water return stream in line 32 can each be passed separately or conjunctively to the water stripper column 30.

The combined product water stream in line 36 includes dilute hydrocarbon oxygenates such as DME, methanol, acetaldehyde, acetone and MEK. The water stripper column 30 separates or strips the oxygenates into a methanol and oxygenate rich stream in an overhead line 44, which is rich in both methanol and at least another oxygenate, and a water rich stripper bottoms stream in a bottoms line 33. The water rich stripper bottoms stream in line 33 is split into a reboil stream in line 37 that is heated and returned to the column, a circulating stripped water stream in line 46 and a net stripper bottoms stream in line 38. The net stripper bottoms stream in line 38 may be split between a lean solvent stream in line 362 and a net stripped water stream in line 49. In one embodiment, the water stripper column 30 temperature may be about 115° C. (239° F.) to about 180° C. (356° F.) at the bottom of the water stripper column and the pressure may be about 75 kPa gauge (11 psig) to about 760 kPa (110 psig) at the overhead of the water stripper column 30.

As disclosed hereinabove in detail, applicants found that a circulating stripped water stream in line 46 from the stripper bottoms stream in line 33 from the separation section 21 of the oxygenate conversion unit 110 can be suitably used for reboiling the second reboiling stream in line 226b in the reboiler 320 from the bottoms stream in the bottoms line 226 of the second distillation column 220. Accordingly, the circulating stripped water stream in line 46 from FIG. 4 is passed to the reboiler 320 for heating the second reboiling stream in line 226b to provide the second reboiled stream in line 322 and a cooled circulating stripped water stream in line 47 as shown in the embodiment of FIGS. 1 and 3. The cooled circulating stripped water stream in line 47 is circulated back to the separation section 21 of the oxygenate conversion unit 110. Alternatively, the circulating stripped water stream in line 46 could preheat the feed to the second distillation column 220 in line 218a in the methanol purification unit 201 as shown in the embodiment of FIG. 2.

The product olefin stream in the product overhead line 40 carries valuable olefinic products which must be recovered. The compression section 80 increases the pressure of the product olefin stream necessary for downstream processing such as used in conventional light olefin recovery units. The compression section 80 may comprise a first knock out drum 82 which separates the product olefin stream into a pressurized first olefin rich stream at a temperature of about 40° C. (104° F.) to about 60° C. (140° F.) and a pressure of about 193 kPa (g) (28 psig) to about 262 kPa (g) (38 psig) in an overhead line 83 and a first aqueous stream rich in oxygenates in a bottoms line 84. The olefin rich stream in the overhead line 83 may be fed to a compressor 85, cooled and directed to a second knockout drum 86. The aqueous stream in the bottoms line 84 is pumped via a manifold line 76 to the return line 32 which returns the water stream with the cooled product water stream in the combined product water stream in line 36 to the water stripper column 30.

The compression section 80 may comprise a second knock out drum 86 which separates the pressurized first olefin rich stream into a second pressurized olefin rich stream at a pressure of about 330 kPa (g) (48 psig) to about 400 kPa (g) (58 psig), and a temperature of about 27° C. (80° F.) to about 54° C. (130° F.) in an overhead line 87 and a second aqueous stream rich in oxygenates in a bottoms line 88. The second olefin rich stream in the overhead line 87 may be fed to a compressor 89, cooled and directed to a third knockout drum 90. The aqueous stream in the bottoms line 88 is passed to the return line 32 via the manifold line 76 which returns the water stream with the cooled product water stream in the combined product water stream in line 36 to the water stripper column 30.

The compression section 80 may comprise a third knock out drum 90 which separates the pressurized second olefin rich stream into a third pressurized olefin rich stream in an overhead line 91 and a third aqueous stream rich in oxygenates in a bottoms line 92. The third olefin rich stream in the overhead line 91 may be fed to the oxygenate absorber column 50. The aqueous stream in the bottoms line 92 is passed to the return line 32 via manifold line 76 which returns the water stream with the warm product water stream in the combined product water stream in line 36 to the water stripper column 30.

Types of suitable compressors may include centrifugal, positive displacement, piston, diaphragm, screw, and the like. In one embodiment, the compressors 85, 89 in the compression section 80 are centrifugal compressors. The final discharge pressure can be between about 1 MPa gauge (145 psig) and about 2 MPa gauge (290 psig). The compressor discharge may be cooled to about ambient temperatures using conventional heat transfer methods.

As illustrated in the FIG. 4 and according to a preferred embodiment, at least a portion of the compressed product stream via the overhead line 91 is contacted in the oxygenate absorber column 50 at effective conditions to absorb at least a quantity of effluent oxygenates with a cooled lean water stream with no water taken directly from the product separator column 24 without prior removal of oxygenates in the water stripper 30. In an exemplary embodiment, a solvent stream in line 102 taken from the cooled circulating stripped water stream in line 47 may be passed to the oxygenate absorber column 50. In an aspect, the solvent stream in line 102 may be further cooled by passing it through a cooler 23 and a cooled solvent stream is passed to the oxygenate absorber column 50. The contacting conducted in the oxygenate absorber column 50 produces an olefin stream with reduced oxygenate content in the overhead line 54 and an oxygenate rich water stream in a bottoms line 52 comprising a quantity of effluent oxygenates. The oxygenate absorber may have operating conditions including a bottoms temperature range of about 30° C. (86° F.) to about 60° C. (140° F.) and an overhead pressure range of about 700 kPa gauge (101 psig) to about 2 MPa gauge (305 psig).

The olefin stream with reduced oxygenate content in the overhead line 54 may be fed to an absorber separator 60 in which a gaseous olefin stream is taken in an overhead line 61 to a third compressor 62 while water is taken in the bottoms line 59 to the manifold line 76. The gaseous olefin stream in line 61 is compressed in the third compressor 62, combined with the stream in a stripper overhead line 71, partially condensed by cooling in the heat exchanger 64, and fed in line 65 to a stripper separator 66. The stripper separator separates an aqueous stream including oxygenates in the boot in line 67 which feeds the manifold line 76, a light olefinic vapor stream in an overhead line 68 comprising C3-olefins and a heavy olefinic liquid stream comprising C4+ olefins in line 69. The heavy olefinic liquid stream in line 69 is stripped in a DME stripper column 70 to remove C3− and lighter vapors in a stripper overhead line 71 from the heavy olefinic liquid stream in the stripper bottoms line 168. A reboil stream may be taken in line 167 from the stripper bottoms and passed to the bottoms of the DME stripper column 70 after reboiling. Most oxygenates will be stripped into the stripper overhead line 71 and be separated after cooling upon recycle to the stripper separator 66. The stripper separator 66 may operate at a temperature of about 15° C. (59° F.) to about 60° C. (140° F.) and a pressure of about 1.7 MPa (g) (250 psig) to about 2.1 MPa (g) (300 psig). The light olefinic vapor stream in the overhead line 68 is scrubbed in a caustic scrubber column 73 by countercurrent contact with a caustic solution from line 42 to absorb acid gases such as carbon dioxide from the light olefinic vapor stream which exits the caustic scrubber 73 in an overhead line 74. In an aspect, the acid gas rich caustic solution exits the scrubber 73 in a bottoms line 44 and may be fed to the water stripper manifold 76.

The scrubbed light olefinic vapor in overhead line 74 may be refrigerated by a refrigerant in a chiller 75 to liquefy part of the light olefinic stream and a cooled light olefinic vapor stream in line 74′ is separated in a drier separator 346 to provide an aqueous stream in line 347 from a boot which is taken to the manifold line 76 and a vaporous light olefin stream comprising C2− hydrocarbons and gases in an overhead line 77 and a liquid light olefin stream in a bottoms line 78 comprising C3+ hydrocarbons. The vaporous light olefin stream in the overhead line 77 is dried in a drier 79a to provide a vaporous product olefin stream in line 112. The liquid light olefin stream in the bottoms line 78 is dried in a drier 79b to provide a liquid product olefin stream in line 114. The product olefin streams in lines 112, 114, and 168 are withdrawn and can be further processed.

The combined product water stream in line 36 includes dilute hydrocarbon oxygenates such as DME, methanol, acetaldehyde, acetone and MEK. The water stripper column 30 separates or strips the oxygenates into a methanol and oxygenate rich stream in an overhead line 44 rich in both methanol and at least another oxygenate and a water rich stream in a bottoms line 33.

The water rich stripper bottoms stream in the bottoms line 33 can be divided into a stripper reboil stream that is reboiled and returned to the water stripping column 30 and a net water rich stripper bottoms stream in line 38. A circulating stripped water stream in line 46 may be taken from the stripper reboil stream in line 37. The net water rich stripper bottoms stream in line 38 may be split into a lean solvent stream provided in line 362 to an extractive distillation column 360 and a remaining stripped water stream in the remaining bottoms line 49. The circulating stripped water stream in line 46 may be passed to the reboiler 310 of the methanol purification section 201 in the embodiment of FIG. 1 or 3 for heating the first reboiling stream in line 218b to provide the first reboiled stream in line 312 and the cooled circulating stripped water stream in line 47. The cooled circulating stripped water stream in line 47 is circulated back to the separation section 21 of the oxygenate conversion unit 110. The circulating stripped water stream in line 46 may alternatively be passed to heat exchanger 219 to preheat the feed in line 218a to the second fractionation column 220 in the embodiment of FIG. 2. The cooled circulating stripped water stream in line 47 can be fed to the quench column 20 in line 19 and to the oxygenate absorber 50 in line 102.

Uncondensed light hydrocarbons can be purged from a receiver overhead line 41 while a methanol and oxygenate rich stream can be removed in a net overhead liquid line 48 and comprise methanol, DME, acetaldehyde, acetone and MEK. A portion of the methanol and oxygenate rich stream can be returned to the water stripper column 30 as reflux.

In one embodiment the water stripper column 30 temperature may be about 115° C. (239° F.) to about 150° C. (302° F.) at the bottom of the water stripper column and the pressure may be about 75 kPa gauge (11 psig) to about 345 kPa (50 psig) at the top of the water stripper column.

The methanol and oxygenate rich stream in overhead liquid line 48 may be fed to the extractive distillation column 360 to separate methanol from at least one other oxygenate. However, the methanol and oxygenate rich stream in overhead liquid line 48 comprises DME which easily separates from methanol. Hence, the methanol and oxygenate rich stream in overhead liquid line 48 may be fed to a DME stripper column 350 to easily remove the DME. The DME stripper column 350 may be in downstream communication with the water stripper column 30. The DME stripper column 350 may separate or strip DME into a DME rich stream in an overhead line 352 and provide a DME lean, methanol and oxygenate rich stream in a bottoms line 354. The DME rich stream in the overhead line 352 may be recycled to the oxygenate conversion reactor 16 in the oxygenate conversion section 11 as reactant feed. A portion of the DME lean, methanol and oxygenate rich stream from the bottoms of the DME stripper column 350 may be reboiled and recycled to the DME stripper column 350. The DME lean, methanol and oxygenate rich stream in bottoms line 354 may be fed to the extractive distillation column 360. The extractive distillation column 360 may be in downstream communication with the water stripper column 30 and upstream of any communication with the product separator column 24 to assure that no inert oxygenates build up in the compression section without an avenue for return to the water stripper column 30. Additionally, in an embodiment, the extractive distillation column 360 may be in downstream communication with the DME stripper column 350.

In one embodiment the DME stripper column 350 temperature may be about 85° C. (185° F.) to about 120° C. (248° F.) at the bottom of the DME striper column and the pressure may be about 75 kPa gauge (11 psig) to about 414 kPa (60 psig) at the top of the column. The DME stripper column 350 may utilize an overhead condenser and receiver separator in addition to or instead of the overhead condenser and receiver 45 for the water stripper column 30 to remove a light hydrocarbon purge. The DME stripper overhead can be recycled to the oxygenate conversion section 11.

The DME lean, methanol and oxygenate rich stream in the net bottoms line 354 may be fed to the extractive distillation column 360 to separate methanol from at least one other hydrocarbon oxygenate and preferably all other hydrocarbon oxygenates. The lean solvent stream in line 362 may also be fed to the extractive distillation column 360 at a location, such as at the top quarter of the column, above a location, such as the middle quarter of the column, at which the DME lean, methanol and oxygenate rich stream in line 354 is fed to the column. The lean solvent stream may be provided in line 362 which may be taken from the water rich stream in the water stripper bottoms line 33.

The flow rate of the lean solvent stream in line 362 to the extractive distillation column 360 should be 1.5 to about 3 times that of the flow rate of oxygenates to the extractive distillation column 360 in the DME lean, methanol and oxygenate rich stream in line 354. The DME, lean, methanol and oxygenate rich stream may also comprise substantial water.

The extractive distillation column 360 produces an oxygenate rich stream in an overhead line 364 comprising at least one oxygenate other than methanol such as acetone, acetaldehyde, MEK, MIPK, DME, ethanol, C3 and C4 alcohols, acetaldehyde, acetic and formic acid and a methanol rich extract stream in a bottoms line 366. A portion of the methanol rich stream in the bottoms line 366 may be reboiled and returned to the extractive distillation column 360. The oxygenate rich stream in the overhead line 364 may be cooled and partially condensed and fed to a extraction receiver 365. Uncondensed light hydrocarbons can be purged in a receiver overhead line 369 while an oxygenate rich stream can be removed in a net overhead liquid stream in line 368 and comprise acetaldehyde, acetone, and MEK. A portion of the oxygenate rich stream can be returned to the extractive distillation column 360 as reflux at a location above the location at which the lean solvent stream in line 362 is added to the extractive distillation column 360. The light hydrocarbon purge(s) in line 369 may be fed to a fuel gas header.

At least 99 wt %, and preferably at least 99.5 wt %, of the hydrocarbon oxygenates other than methanol, DME and ethanol fed to the extractive distillation column 360 may be recovered in the oxygenate rich stream in the overhead line 364 of the extractive distillation column 360 and the oxygenate rich stream in the net overhead liquid line 368 of the extraction receiver 365. At least 95 wt %, and preferably at least 99.5 wt %, of the methanol may be recovered in the methanol and water rich stream in the net bottoms line 366.

The extractive distillation column 360 may have operating conditions including a bottoms temperature in the range of about 75° C. (167° F.) to about 150° C. (302° F.) and an overhead pressure in the range of about 75 kPa gauge (11 psig) to about 200 kPa gauge (29 psig). The extractive distillation column 360 may be in downstream communication with the overhead line 44 of the water stripper column 30 and with a remaining bottoms line 49 of the water stripper column 30.

The recovered methanol is an MTO reactant that can be recycled to the MTO reactor or oxygenate conversion reactor 16, but it is not desirable to recycle the water with the methanol. Hence, the methanol and water rich stream in the net bottoms line 366 may be fed to a methanol stripper column 370 to separate a methanol rich stream in an overhead line 372 from a final water rich stream in a bottoms line 374. The methanol rich stream in the overhead line 372 may then be recycled to the MTO reactor 16 without oxygenates that can otherwise build up in the process and apparatus 110. A portion of the final water rich stream in the bottoms line 374 may be reboiled and recycled to the methanol stripper column 370. The final water rich stream in the net bottoms line 374 may be forwarded to water treatment in line 375 along with an unrecycled portion of the water rich stream in the net stripped water stream in the remaining bottoms line 49 from the water rich stripper bottoms stream in line 33.

### EXAMPLE

A simulation study was performed to demonstrate the water rich stream from the separation section 21 of the oxygenate conversion unit 110 can be used for providing the reboiler duty of the first distillation column 310 and the second distillation column 320. The Table below summarizes the exchanger duties, stream flow rates, and temperatures for reboilers 310 and 320.

| | | TABLE | | | |
|---|---|---|---|---|---|
| | | Streams | | | |
| | | Product | Cooled | First | First |
| | | Water | Product | Reboiling | Reboiled |
| | | Stream | Water | Stream | Stream |
| Reboiler | | (26) | Stream (27) | (218b) | (312) |
| Reboiler | Temperature | 122 | 105 | 99 | 99 |
| 310 | (° C., ° F.) | 252 | 221 | 211 | 211 |
| | Flow Rate | 680000 | 680000 | 140673 | 140673 |
| | (kg/hr., lbs./hr.) | 1500000 | 1500000 | 310130 | 310130 |

| Reboiler (310) duty | | 13.8, 47.2 (MW, mmbtu/hr.) | | | |
|---|---|---|---|---|---|
| | | Circulating | Cooled | | |
| | | Stripped | Circulating | Second | Second |
| | | Water | Stripped | Reboiling | Reboiled |
| | | Stream | Water | Stream | Stream |
| | | (46) | Stream (47) | (226b) | (322) |
| Reboiler | Temperature | 174 | 139 | 134 | 134 |
| 320 | (° C., ° F.) | 346 | 283 | 273 | 273 |
| | Flow Rate | 150267 | 150267 | 522113 | 522113 |
| | (kg/hr., lbs./hr.) | 331281 | 331281 | 1151061 | 1151061 |

| | |
|---|---|
| Reboiler (320) duty | 6.3, 21.4 (MW, mmbtu/hr.) |

The product water stream in line 26 provided all the required duty for the reboiler 310. The circulating stripped water stream in line 46 also met some of the duty requirement for the reboiler 320.

### Specific Embodiments

While the following is described in conjunction with specific embodiments, it will be understood that this description is intended to illustrate and not limit the scope of the preceding description and the appended claims.

A first embodiment of the present disclosure is an integrated process for producing light olefins comprising passing a syngas stream to a methanol synthesis reactor to provide a reactor effluent comprising methanol; separating the reactor effluent into a vapor stream and a liquid stream comprising methanol; passing the liquid stream comprising methanol to a methanol purification section comprising a first distillation column to provide a methanol product stream; passing at least a portion of the methanol product stream to an oxygenate conversion unit to provide an effluent comprising olefins; and separating light olefins from the effluent comprising olefins in a separation section of the oxygenate conversion unit, wherein heat to the first distillation column is provided from the separation section of the oxygenate conversion unit. An embodiment of the present disclosure is one, any or all of prior embodiments in this paragraph up through the first embodiment in this paragraph, wherein the methanol synthesis section comprises a first methanol converter and a second methanol converter. An embodiment of the present disclosure is one, any or all of prior embodiments in this paragraph up through the first embodiment in this paragraph further comprising passing the syngas stream to the first methanol converter to provide a first reactor effluent comprising methanol; separating the first reactor effluent into a first vapor stream and a first liquid stream; passing the first vapor stream to the second methanol converter to provide a second reactor effluent comprising methanol; separating the second reactor effluent into a second vapor stream and a second liquid stream; separating the first liquid stream and the second liquid stream into an overhead stream comprising lights and a bottoms stream comprising crude methanol; and passing the bottoms stream comprising crude methanol to the methanol purification unit. An embodiment of the present disclosure is one, any or all of prior embodiments in this paragraph up through the first embodiment in this paragraph wherein the step of passing the liquid stream comprises passing the liquid stream comprising methanol to the first distillation column to provide a first distillation column overhead stream and a first distillation column bottoms stream; separating the first distillation column bottoms stream into a first reboiling stream and a first distillation column effluent stream; heating the first reboiling stream in a reboiler of the first distillation column with a product water stream taken from the separation section to provide a first reboiled stream and a cooled product water stream; passing the first reboiled stream to the first distillation column; passing the first distillation column effluent stream to a second distillation column to provide a second distillation column overhead stream and a second distillation column bottoms stream; separating the second distillation column bottoms stream into a second reboiling stream and a second distillation column effluent stream; heating the second reboiling stream in a reboiler of the second distillation column with a stripped water stream of the separation section to provide a second reboiled stream and a cooled stripped water stream; and passing a second reboiled stream to the second distillation column. An embodiment of the present disclosure is one, any or all of prior embodiments in this paragraph up through the first embodiment in this paragraph further comprising separating the first distillation column overhead stream to provide a first reflux stream and a first overhead liquid stream comprising the methanol product. An embodiment of the present disclosure is one, any or all of prior embodiments in this paragraph up through the first embodiment in this paragraph wherein the stripped water stream is obtained from the cooled product water stream. An embodiment of the present disclosure is one, any or all of prior embodiments in this paragraph up through the first embodiment in this paragraph further comprising heating the liquid stream comprising methanol before passing to the first distillation column with one or both of the stripped water stream and the product water stream. An embodiment of the present disclosure is one, any or all of prior embodiments in this paragraph up through the first embodiment in this paragraph further comprising passing the second distillation column effluent stream to a third distillation column to provide the methanol product stream. An embodiment of the present disclosure is one, any or all of prior embodiments in this paragraph up through the first embodiment in this paragraph wherein the methanol purification unit comprises two stripper columns. An embodiment of the present disclosure is one, any or all of prior embodiments in this paragraph up through the first embodiment in this paragraph further comprising passing the effluent comprising olefins to a quench column in the separation section to provide a quenched effluent stream; passing the quenched effluent stream to a product separator column in the separation section to provide an overhead stream comprising olefins and a bottoms water stream; taking the product water stream from the bottoms water stream; passing the product water stream to the reboiler of first distillation column; and passing a cooled product water stream to the separation section. An embodiment of the present disclosure is one, any or all of prior embodiments in this paragraph up through the first embodiment in this paragraph further comprising passing the cooled product water stream, an overhead water stream and an intermediate water stream to a water stripper column of the separation section to provide an oxygenate rich overhead stream and a water rich bottoms stream passing at least a portion of the water rich bottoms stream to the reboiler of the first distillation column; and passing a cooled water rich bottoms stream to a quench column of the separation section. An embodiment of the present disclosure is one, any or all of prior embodiments in this paragraph up through the first embodiment in this paragraph wherein the intermediate water stream is provided from a product separator column and the overhead water stream is provided from a compression section of the separation section.

A second embodiment of the present disclosure is an integrated process for producing light olefins comprising passing a syngas stream to a methanol synthesis reactor to provide a reactor effluent comprising methanol; separating the reactor effluent into a vapor stream and a liquid stream comprising methanol; passing the liquid stream comprising methanol to a methanol purification section comprising a first distillation column and a second distillation column to provide a methanol product stream; and passing at least a portion of the methanol product stream to an oxygenate conversion unit comprising a separation section for separating light olefins to provide an effluent comprising olefins, wherein heat to the first distillation column and the second distillation column is provided from a water stream taken from the separation section. An embodiment of the present disclosure is one, any or all of prior embodiments in this paragraph up through the first embodiment in this paragraph wherein the step of passing the liquid stream comprises passing the liquid stream comprising methanol to the first distillation column to provide a first distillation column overhead stream and a first distillation column bottoms stream; separating the first distillation column bottoms stream into a first reboiling stream and a first distillation column effluent stream; heating the first reboiling stream in a reboiler of the first distillation column with a product water stream taken from the separation section to provide a first reboiled stream and a cooled product water stream; passing the first reboiled stream to the first distillation column; passing the first distillation column effluent stream to the second distillation column to provide a second distillation column overhead stream and a second distillation column bottoms stream; separating the second distillation column bottoms stream into a second reboiling stream and a second distillation column effluent stream; heating the second reboiling stream in a reboiler of the second distillation column with a stripped water stream of the separation section to provide a second reboiled stream and a cooled stripped water stream; and passing a second reboiled stream to the second distillation column. An embodiment of the present disclosure is one, any or all of prior embodiments in this paragraph up through the first embodiment in this paragraph wherein the stripped water stream is obtained from the cooled product water stream. An embodiment of the present disclosure is one, any or all of prior embodiments in this paragraph up through the first embodiment in this paragraph further comprising passing the second distillation column effluent stream to a third distillation column to provide a methanol product stream.

A third embodiment of the present disclosure is a process for producing light olefins comprising passing a syngas stream to a methanol synthesis reactor to provide a reactor effluent comprising methanol; separating the reactor effluent into a vapor stream and a liquid stream comprising methanol; passing the liquid stream comprising methanol to a methanol purification section comprising a first distillation column and a second distillation column to provide a methanol product stream; and wherein a first reboiling stream of the first distillation column is heated in a reboiler of the first distillation column with a product water stream taken from the separation section and a second reboiling stream of the second distillation column is heated in a reboiler of the second distillation column with a stripped water stream of the separation section

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. An integrated process for producing light olefins comprising:
passing a syngas stream to a methanol synthesis reactor to provide a reactor effluent comprising methanol;
separating the reactor effluent into a vapor stream and a liquid stream comprising methanol;
passing the liquid stream comprising methanol to a methanol purification section comprising a first distillation column to provide a methanol product stream;
passing at least a portion of the methanol product stream to an oxygenate conversion unit to provide an effluent comprising olefins; and
separating light olefins from said effluent comprising olefins in a separation section of said oxygenate conversion unit,
wherein heat to said first distillation column is provided from the separation section of said oxygenate conversion unit.

2. The process of claim 1, wherein the methanol synthesis section comprises a first methanol converter and a second methanol converter.

3. The process of claim 2 further comprising:
passing the syngas stream to the first methanol converter to provide a first reactor effluent comprising methanol;
separating the first reactor effluent into a first vapor stream and a first liquid stream;
passing the first vapor stream to the second methanol converter to provide a second reactor effluent comprising methanol;
separating the second reactor effluent into a second vapor stream and a second liquid stream;
separating the first liquid stream and the second liquid stream into an overhead stream comprising lights and a bottoms stream comprising crude methanol; and
passing the bottoms stream comprising crude methanol to said methanol purification unit.

4. The process of claim 1 wherein the step of passing the liquid stream comprises:
passing the liquid stream comprising methanol to the first distillation column to provide a first distillation column overhead stream and a first distillation column bottoms stream;
separating the first distillation column bottoms stream into a first reboiling stream and a first distillation column effluent stream;
heating said first reboiling stream in a reboiler of the first distillation column with a product water stream taken from the separation section to provide a first reboiled stream and a cooled product water stream;
passing said first reboiled stream to said first distillation column;
passing the first distillation column effluent stream to a second distillation column to provide a second distillation column overhead stream and a second distillation column bottoms stream;
separating the second distillation column bottoms stream into a second reboiling stream and a second distillation column effluent stream;
heating said second reboiling stream in a reboiler of the second distillation column with a stripped water stream of the separation section to provide a second reboiled stream and a cooled stripped water stream; and
passing a second reboiled stream to said second distillation column.

5. The process of claim 4 further comprising:
separating said first distillation column overhead stream to provide a first reflux stream and a first overhead liquid stream comprising the methanol product.

6. The process of claim 4 wherein the stripped water stream is obtained from said cooled product water stream.

7. The process of claim 4 further comprising heating said liquid stream comprising methanol before passing to the first distillation column with one or both of the stripped water stream and the product water stream.

8. The process of claim 4 further comprising passing the second distillation column effluent stream to a third distillation column to provide said methanol product stream.

9. The process of claim 1 wherein the methanol purification unit comprises two stripper columns.

10. The process of claim 4 further comprising:
passing said effluent comprising olefins to a quench column in said separation section to provide a quenched effluent stream;
passing said quenched effluent stream to a product separator column in said separation section to provide an overhead stream comprising olefins and a bottoms water stream;
taking the product water stream from the bottoms water stream;
passing the product water stream to the reboiler of the first distillation column; and
passing a cooled product water stream to the separation section.

11. The process of claim 4 further comprising:
passing the cooled product water stream, an overhead water stream and an intermediate water stream to a water stripper column of said separation section to provide an oxygenate rich overhead stream and a water rich bottoms stream:
passing at least a portion of said water rich bottoms stream to the reboiler of said second distillation column; and
passing a cooled water rich bottoms stream to a quench column of said separation section.

12. The process of claim 11 wherein the intermediate water stream is provided from a product separator column and the overhead water stream is provided from a compression section of said separation section.

13. The process of claim 12 further comprising:
providing an intermediate liquid stream from said product separator column;
separating the intermediate liquid stream into an intermediate reflux stream and the intermediate water stream; and
passing the overhead stream comprising olefins to the compression section to separate olefins and provide the overhead water stream.

14. The process of claim 1 wherein the methanol purification section comprises a third distillation column.

15. The process of claim 14 further comprising:
fractionating a second distillation column effluent stream in the third distillation column to provide a third distillation column overhead stream comprising methanol; and
separating the third distillation column overhead stream to provide a third reflux stream and a methanol stream;
heating a third reboiling stream in a reboiler of the third distillation column with a second distillation column overhead stream to provide a third reboiled stream; and
passing the third reboiled stream to the third distillation column.

16. An integrated process for producing light olefins comprising:
passing a syngas stream to a methanol synthesis reactor to provide a reactor effluent comprising methanol;
separating the reactor effluent into a vapor stream and a liquid stream comprising methanol;
passing the liquid stream comprising methanol to a methanol purification section comprising a first distillation column and a second distillation column to provide a methanol product stream; and
passing at least a portion of the methanol product stream to an oxygenate conversion unit comprising a separation section for separating light olefins to provide an effluent comprising olefins,
wherein heat to said first distillation column and the second distillation column is provided from a water stream taken from the separation section.

17. The process of claim 16 wherein the step of passing the liquid stream comprises:
passing the liquid stream comprising methanol to the first distillation column to provide a first distillation column overhead stream and a first distillation column bottoms stream;
separating the first distillation column bottoms stream into a first reboiling stream and a first distillation column effluent stream;
heating said first reboiling stream in a reboiler of the first distillation column with a product water stream taken from the separation section to provide a first reboiled stream and a cooled product water stream;
passing said first reboiled stream to said first distillation column;
passing the first distillation column effluent stream to said second distillation column to provide a second distillation column overhead stream and a second distillation column bottoms stream;
separating the second distillation column bottoms stream into a second reboiling stream and a second distillation column effluent stream;
heating said second reboiling stream in a reboiler of the second distillation column with a stripped water stream of the separation section to provide a second reboiled stream and a cooled stripped water stream; and
passing a second reboiled stream to said second distillation column.

18. The process of claim 17 wherein the stripped water stream is obtained from said cooled product water stream.

19. The process of claim 17 further comprising passing the second distillation column effluent stream to a third distillation column to provide a methanol product stream.

20. An integrated process for producing light olefins comprising:
passing a syngas stream to a methanol synthesis reactor to provide a reactor effluent comprising methanol;
separating the reactor effluent into a vapor stream and a liquid stream comprising methanol;
passing the liquid stream comprising methanol to a methanol purification section comprising a first distillation column and a second distillation column to provide a methanol product stream; and
wherein a first reboiling stream of the first distillation column is heated in a reboiler of the first distillation column with a product water stream taken from the separation section and a second reboiling stream of the second distillation column is heated in a reboiler of the second distillation column with a stripped water stream of the separation section.
